(19) United States
(12) Patent Application Publication
(10) Pub. No.: US 2021/0322653 A1
(43) Pub. Date: Oct. 21, 2021

(54) ANTIMICROBIAL IMPLANTABLE MEDICAL DEVICES

(71) Applicant: CuPro Technologies LLC, Salt Lake City, UT (US)

(72) Inventor: Giridhar Thiagarajan, Salt Lake City, UT (US)

(21) Appl. No.: 17/312,579

(22) PCT Filed: Dec. 10, 2019
(86) PCT No.: PCT/US2019/065474

§ 371 Date: Jun. 10, 2021

Related U.S. Application Data
(60) Provisional application No. 62/777,486, filed on Dec. 10, 2018.

(51) Int. Cl. A61L 31/16; A61F 11/00; A61L 29/10; A61L 31/08; A61L 29/16

(52) U.S. Cl. CPC A61L 31/16; A61F 11/002; A61L 29/106; A61L 31/088; A61L 29/16; A61L 2420/02; A61L 2300/404; A61L 2400/18; A61L 2430/14; A61L 2430/32; A61L 2300/102

(57) ABSTRACT

The present disclosure is drawn to antimicrobial implantable medical devices, and can include an implantable medical device, and an antimicrobial metal applied to an exterior surface of at least a portion of the implantable medical that is positionable within a body tissue or traverses the body tissue when surgically placed (using surgical instruments beyond merely a needle or catheter port) for implantation.

### BACKGROUND

Long term medical devices of various types that interface with the body can be susceptible to infection. Transcutaneous and internally-positioned medical devices are often susceptible to microbial colonization, which may often originate at the site of transcutaneous access and often progress inwards into nutrient rich environments of the body. However, body cavity infections (where an implant is being held within the body), though less common, can sometimes be even more problematic, even though they statistically occur less frequently. This is because they may be more difficult to access (or even discover) without surgical intervention or investigation, as such infections can occur well beneath the body surface. Also, these body cavities are sparsely perfused by blood and hence immune surveillance is less prevalent and slower. These types of infections can manifest systemic symptoms eventually but the infection is at an advanced stage at this point and difficult to treat. Regardless, both sources of infection, e.g., transcutaneous infection and body cavity infection, are an issue in the field of medical implants, particularly for medical implants that are intended for long term use, e.g., more than a few weeks, months, years, or sometimes a life-time. The challenge with such medical device infections is a combination of factors such as introduction of a microbial load during surgical intervention. In the case of transcutaneous implants, continual existence of the skin breach that may not be effectively closed up by wound healing responses due to the presence of the implant.

### BRIEF DESCRIPTION OF THE DRAWINGS

FIG. 1 schematically illustrates an example antimicrobial implantable medical device including an example ear tube coated, such as at least on the external surface, with an antimicrobial metal layer in accordance with the present disclosure;

FIG. 2 schematically illustrates an example antimicrobial implantable medical device including an example external ventricular drainage (EVD) device coated on the external surface with an antimicrobial metal layer in accordance with the present disclosure;

FIG. 3 schematically illustrates an example antimicrobial implantable medical device including an example ventricular assist device (VAD) with an antimicrobial metal layer applied to the driveline as well as to portions of the pump in accordance with the present disclosure;

FIG. 4 is a bar graph depicting the antimicrobial performance of a copper plated silicone tympanostomy tube (ear tube) in terms of log-scale reduction of Staphylococcus aureus compared to an otherwise identical uncoated ear tube, and further in comparison to a commercially available antimicrobial ear tube;

FIG. 5 is a bar graph depicting the antimicrobial performance of a copper plated FLP tympanostomy tube (ear tube) in terms of log-scale reduction of Staphylococcus aureus compared to an otherwise identical uncoated ear tube, and further in comparison to a commercially available antimicrobial ear tube;

FIG. 6 is a bar graph depicting the antimicrobial performance of a copper plated External Ventricular Drainage (EVD) device made of silicone in terms of log-scale reduction of Staphylococcus epidermidis compared to an otherwise identical uncoated EVD, and further in comparison to commercially available antibiotic coating systems;

FIG. 7 is a bar graph depicting the antimicrobial performance of a copper plated External Ventricular Drainage (EVD) device made of silicone in terms of log-scale reduction of Staphylococcus aureus compared to an otherwise identical uncoated EVD, and further in comparison to two commercially available antibiotic coating systems; and

FIG. 8 is a bar graph depicting the antimicrobial performance of a copper plated External Ventricular Drainage (EVD) in terms of log-scale reduction or Staphylococcus aureus compared to an otherwise identical uncoated EVD, and further in comparison to a commercially available antibiotic coating system.

FIG. 9 is a bar graph depicting results from an example ex vivo animal study relating to animal tolerance and antimicrobial efficacy compared to a control and a commercial product.

### DETAILED DESCRIPTION

One approach to providing antimicrobial properties to an implantable medical device is to coat a surface of the medical device with an anti-infective compound where it may be susceptible to infection, such as bacterial colonization. One of the technical challenges with many antibiotic coatings on medical devices, however, is their lack of long term effectiveness. For example, drug-eluting coatings often deplete quickly in the body due to limited availability of drug reservoir after which they can be ineffective, e.g., a thin coating does not provide a large reservoir, and on the other hand, non-eluting passive coatings tend to bio-foul fairly quickly. This can limit the antimicrobial effect of such technologies to only a few hours, a few days, but typically not in the order of weeks. As long term devices, e.g., 5 days to 5 years, 1 week to 2 years, 2 weeks to 2 year, 4 weeks to 2 years, 1 week to 1 year, 2 weeks to 1 year, 4 weeks to 1 year, 1 week to 6 months, 2 weeks to 6 months, 4 weeks to 6 months, etc., would benefit greatly from a more durable and/or longer lasting form of protection to avoid clinical complications such as mortality and morbidity, implantable medical devices of the present disclosure can provide, in some cases, antimicrobial benefits for either short or long term use.

Thus, the present disclosure is drawn to the application of an antimicrobial metal, which includes elemental metal coatings or metal alloy coatings, provided there is at least one metal present in the metal alloy at a high enough concentration to exhibit antimicrobial properties at a location where the medical device is in contact with body tissue susceptible to infection. In one example, the antimicrobial metal coating can be applied by electroless deposition or other technique, such as sputtering, weaving, dip coating, etc. However, the coating of rubbers, plastics, ceramics, metals, and other materials used for medical devices can be coated effectively using electroless deposition or other electroplating techniques. These coating technologies can be particularly useful in applications where the antimicrobial metal to be applied is intended to include a non-leaching metal, such as the contact killing metal copper. In further detail, though any antimicrobial metal can be used, such as silver, zinc, gold, tin, or alloys thereof, in one specific example, the antimicrobial metal can be or include a contact killing metal, such as copper.

For further clarity, the present disclosure is not directed to coating of the interior of medical devices that do not contact body tissue, e.g., luminal coatings within catheters, for protecting against infection related to blood or other flow of fluid through a medical device, but rather, is directed to protecting medical devices from infections where the medical device contacts body tissue other than luminal fluids on the external surface. The term “tissue” herein excludes blood or urine but rather includes solid body tissue and associated fluid that comes in contact with an exterior surface of a medical device. With this understanding, implementation or use of the medical devices of the present disclosure does not preclude the use of implantable medical device coatings within lumens of the implantable medical device, including even antimicrobial metal layers applied thereto. However, to the extent those types of coatings are used intraluminally, they may or may not be used in conjunction with the exterior antimicrobial metal coatings of the present disclosure without impacting the characteristics of the external antimicrobial metal layers of the present disclosure.

An “implantable medical device” can be defined herein to include a device assembly or merely a single device assembly part that may be coated with the antimicrobial metal coatings of the present disclosure. For example, Ventricular Assist Devices (VADs), often referred to as a “heart pump” or a Left Ventricular Assist Device (LVAD), includes multiple modular parts that are assembled together. One of those parts is known as a driveline, which is a transcutaneous line that electrically connects a controller on the outside of the body to a pump on the inside of the body at a body cavity near the heart. Thus, the VAD as an assembly can be considered to be a medical device as defined herein, but the driveline per se is also considered to be a medical device in and of itself.

In further detail, “implantable medical devices” as described herein exclude medical devices that routinely access the lumen of a subject by a non-surgical procedure, such as by access through a natural body lumen, e.g. urethral access, vaginal access, oral access, e.g., oral or nasal tracheal tube, or routine needle or catheter port access through the skin and into a luminal vessel. Overall, a vascular access catheter (needle or catheter port access), such as a peripherally inserted central catheter (PICC), a central venous catheter (CVC), a hemodialysis catheter (HD), an arterial catheter, etc., an intrauterine device (IUD) (vaginal access), a urinary catheter (urethral access), an endotracheal tube, an endoscope (colon, nasal, oral access) are not considered to be an implantable medical device as defined herein, even if they remain in the body for an extended period of time. These types of medical devices that are excluded from the “implantable medical device” definition herein can be referred to herein collectively as “luminal medical devices,” as they are either inserted luminally into the body, or the lumen of the body is accessed routinely without surgical incision, e.g., needle or catheter port. In further detail, simple surgical fasteners, such as pins, wires, staples, screws, nails, including those used in orthopedics and other surgeries are also not considered to be implantable medical devices herein due to their simplicity. However, to the extent that they may be used in the context of a larger implantable medical device, they may be likewise coated along with the larger/bulkier and more complex medical device such as an orthopedic prosthetic, e.g., joint replacement prosthetic, bone replacement prosthetic, prosthetic plates specialized for specific anatomical structures, prosthetic support rods other than fasteners, prosthetic devices that have an internal and an external component, etc.

On the other hand, the implantable medical devices described herein are directed toward medical devices that are often extraluminal within the body, but in cases where they do access a body lumen, e.g., tympanostomy tubes (accessing the tympanic membrane through the external auditory canal), they do so in connection with surgical access and implantation. Thus, the implantable medical devices often include other overlapping features, such as not dwelling in the vasculature or urinary tract, and these devices often encounter low fluid (liquid) flow rates around the device (lower than blood flow or urine flow rates).

A few example “implantable medical devices” that may include an added risk of infection due to surgical or implantation complications that can be coated with antimicrobial metal as disclosed herein include, but are not limited to, shunts such as cerebral shunts; ventricular drainage (EVD) devices; ventricular assist devices (VADs) including the transcutaneous drivelines and internally implanted pumps; tympanostomy tubes (ear tubes); sensory or neurological implants such as neurotransmitters; orthopedic implants; surgically implanted feeding tubes provided they surgically cross tissue, e.g. gastric feeding tubes (G-tubes), gastrojejunal feeding tubes, or jejunal feeding tubes; transcutaneous tracheal tubes; surgical meshes, e.g., hernia mesh; transcutaneous sensors, e.g., glucose sensor; implantable sinus drainage devices (that are at least partially implanted within tissue); etc.; to name a few.

As another point of distinction regarding the antimicrobial implantable medical devices, methods, and systems in accordance with the present disclosure, unlike catheters, many of these devices are not attached or used with infusion or aspiration ports that introduce intraluminal pressure (positive or negative) when in operation. Shunts, tympanostomy tubes, electrical power or control lines, implants, etc., are often implanted surgically across tissue or within the body of a subject to operate based on natural fluid flow, electrical control, structural support, etc., without the use of a pump, aspirator, syringe, etc., when in use. These devices are intended to sit within tissue without potential or possible disruptive pressure being applied when in use, for example. Thus, in one example, the implantable medical device in operation includes a lumen that is not subjected to externally applied positive or negative pressure while in use after implantation.

In accordance with this, the present disclosure is drawn to an antimicrobial implantable medical device that can include an implantable medical device, and an antimicrobial metal applied to an exterior surface of at least a portion of the implantable medical device that is positionable within the body tissue or traverses the body tissue when surgically placed for implantation, e.g., by a procedure including a tissue incision, at least partially within the body of a subject. The antimicrobial metal applied to the exterior surface can be positioned at a location in contact with a location of surgical tissue incision or can be implanted beneath or adjacent to the location of surgical incision, for example. The antimicrobial metal can be an antimicrobial metal coating applied by any of a number of coating technologies, such as electroless deposition, or can be applied to a surface by other methodologies, such as by application of a metal filament or fiber to a surface, such as by weaving. The implantable medical device can be at a location where the antimicrobial implantable medical device is within or traverses the body of the subject, in one example, and the antimicrobial metal can be at least 0.5 cm on one side of the body tissue (when within the tissue or traversing the tissue), or at least 0.5 cm on both sides of the body tissue (when traversing the tissue). In other examples, the antimicrobial metal can be independently at least 1 cm, at least 2.5 cm, at least 5 cm, or at least 10 cm on one or both sides of the body tissue. The term “traverses” herein refers to the crossing of a single body tissue from one side to the other, such as skin, an organ wall, the tympanic membrane, etc. Traversing the tissue includes crossing the tissue at the site of an incision and not through a natural body lumen or via needle or catheter port entry, e.g., incisions occurring across the skin, muscle, intestines, tympanic membrane, etc. The term “tissue” is not used so specifically that it refers to a single tissue type or another tissue type, but rather a tissue structure as may define an organ, e.g., stomach, heart, skin, intestines, etc., or the structural components of the musculoskeletal system, e.g., bone, muscles, ligaments, etc.

In another example, a method of ameliorating implantable medical device-related infections can include surgically placing an antimicrobial implantable medical device of the present disclosure at least partially within the body of a subject. The antimicrobial metal of the antimicrobial implantable medical device can be positioned for implantation at a location within a body tissue or traversing the body tissue. In one specific example, the method can include applying an electrical potential to the antimicrobial metal while the metal layer is positioned at the location. In another specific example, the antimicrobial implantable medical device can be a transcutaneous device, and the method can further include leaving a margin of at least 0.5 cm, at least 1 cm, at least 2.5 cm, at least 5 cm, or at least 10 cm outside of the body beyond the location of insertion through the cutaneous tissue.

In another example, a method of manufacturing an antimicrobial implantable medical device can include electroplating/electroless deposition on an implantable medical device as described and defined herein at least at a location where the implantable medical device is to be positioned within a body tissue or traversing the body tissue for implantation. In one specific example, the electroless deposition can be carried out using a divalent copper salt source material which in solution is reduced to metallic copper in the presence of a reducing agent which in turn gets oxidized and the metallic copper atoms are deposited on any surface in the bath including the medical device surfaces thus generating a antimicrobial metal layer. Other metal sources can be used for other metals or metal alloys. In one example, the medical device can have a polymer surface and the method can further include the preliminary step of introducing surface roughness to the polymer surface. Introducing surface roughness, for example, can include chemical etching, mechanical abrasion, physical etching, plasma treatment, etc.

When discussing the medical devices or methods herein, relative details from a discussion of either is considered applicable to the other, whether or not they are explicitly discussed in the context of that example. Thus, for example, in discussing tympanostomy tubes in the context of the medical devices herein, such disclosure is also related to any methods and/or systems also included herein, and vice versa, etc.

Turning now to the antimicrobial metal that is applied to or coated on the medical devices as described herein, examples of metals that can be used include copper, silver, zinc, gold, or alloys thereof. The term “alloys” includes various combinations of two or more of copper, silver, zinc, or gold, but can also include alloys of one or more of these metals with any other metal(s) or non-metal(s) that may provide a therapeutic or other practical property. For example, as copper oxidizes, copper can be alloyed with another metal, such as silver, zinc, and/or gold, but may also be alloyed with metals that may not be necessarily antimicrobial in nature. Examples of other metals that can be alloyed with copper include iron, nickel, aluminum, etc., for the purpose of slowing or preventing oxidation, or for some other therapeutic or practical purpose, e.g., enhanced metal adherence to the medical device surface material, modification of metallurgic properties such as flexibility and/or resilience, etc. As copper is a good metal for providing anti-infective properties due to its ability to contact kill without ion diffusion into or around neighboring tissue, if copper is used, it can be included in the alloy as a substantial portion, e.g., greater than 50 wt %, and a lesser proportion of other metals (or non-metals) may be included to contribute to reduction in copper oxidation, to contribute to antimicrobial effect, e.g., silver, zinc, and/or gold, to contribute to another therapeutic effect, to address a manufacturing concern, to enhance or improve a metal alloy physical property, e.g., flexibility, resilience, malleability, medical device adhesion, etc. Thus, if a copper alloy is used, in one example, the copper can be present in the alloy at from 50 wt % to 99 wt %, from 50 wt % to 95 wt %, from 50 wt % to 90 wt %, from 50 wt % to 80 wt %, 55 wt % to 99 wt %, from 55 wt % to 95 wt %, from 55 wt % to 90 wt %, from 55 wt % to 80 wt %, 60 wt % to 99 wt %, from 60 wt % to 95 wt %, from 60 wt % to 90 wt %, from 60 wt % to 80 wt %, or from 55 wt % to 70 wt %.

In accordance with the present disclosure, a metal based electroplating/electroless deposition technology can be used to address the persistent problem of infections associated with medical devices. The design of this active technology will have long-term, broad spectrum efficacy without relying on release/elution of an active agent. This technology can potentially be applied to the external surface of a wide range of transcutaneous and implantable medical devices.

In more detail with respect to copper, in addition to the cationic nature of copper ions (Cu²⁺) that cause them to bind or become attracted to negatively charged protective cell wall components and obliterate or otherwise disrupt or damage the cell membrane or wall, e.g., bacteria or other microbes, as mentioned briefly, copper can also kill microbes by contact with the elemental metal or with an alloy of the elemental metal (rather than by ions that slowly diffuse into solution over time). Thus, copper in particular can be a good material for use in the coating on the medical device exterior surface because it can kill pathogens continuously upon surface contact where the medical device is directly interfacing with the body tissue to actively reduce microbial colonization at the interface, regardless of ion diffusion. This can occur by virtue of the charge density where the microbe contacts the copper or copper alloy, which may cause membrane damage, nucleic acid damage, and/or generation of a reactive oxygen species that may be detrimental to the microbe. Thus, copper ions do not necessarily need to diffuse or leach out from the surface of the implantable medical device and into the fluid to kill microbes in order to have a good anti-infective effect, though some diffusion around the medical device can provide an added area of protection over time (the time it takes to diffuse outward). Instead, the native elemental or alloyed copper surface can possess active antimicrobial properties that can prevent colonization, and furthermore, the diffusion of ions out into the tissue via any fluid therebetween can enhance further the antimicrobial effect. Because of the contact killing nature of copper in particular, this allows elemental or alloyed copper coatings to last long-term without depletion or consumption of the killing effectiveness. In still further detail, an additional benefit of using a metal with contact killing properties, e.g., copper, is that it may not expose the subject to undesired high dosages of copper, as may be the case if treating tissue with a more eluting-type coating.

The use of a thin coating of copper for use on implantable medical devices may not have been considered previously, particularly for implantable medical devices as defined herein, as an antimicrobial candidate for coating because with some implantable medical devices, there may be a desired degree of flexibility that has been thought to be desirable. A substantially thick copper coating, or even polymeric materials compounded with high concentrations of copper salts in the resin, can become quite rigid. On the other hand, a thin copper-eluting coating may not cause the medical device to be overly rigid, but as a conventional eluting coating, the same issues with respect to antimicrobial agent depletion due to elution from the thin coating may not be more effective than antibiotic coatings, as the minimum effective concentration (MEC) of the copper in such a coating may not be maintainable over a longer period of time to be effective for even a week, for example.

Thus, in accordance with examples of the present disclosure, an antimicrobial metal can be coated as a thin layer, e.g., from 0.0001 μm to 50 μm, from 0.0005 μm to 1 μm, or from 0.001 μm to 0.1 μm, on a medical device to provide an antimicrobial layer thereon. The metal can be as described previously, and can include copper, silver, zinc, gold, or an alloy thereof. In one example, the metal can include copper, which is a contact killer. In still further detail, the metal coating can be a copper or copper alloy layer on a surface of the implantable medical device surface, that can be effectively non-leaching (particularly with copper), and which can retain its contact killing properties over a long period of time, e.g., 1 year or longer. By referring to some antimicrobial metals (typically electroless deposition layers) as “non-leaching” or “non-eluting,” this does not infer that no copper ions or other metal ions that may be present, such as in a copper alloy, elute out into the immediately surrounding tissue at all, but rather, the term non-leaching or non-eluting is defined herein as eluting less than 10 (parts per million) ppm, less than 5 ppm, less than 1 ppm, or less than 100 parts per billion (ppb) of a respective elemental metal, by weight, out from the coating into the surrounding tissue. There can be coatings, for example, where one metal may be non-leaching and the other metal may exceed these limits, but that may be acceptable as determined on a case by case basis based on the total metal content of the specific metal and the impact that metal may have on surrounding body tissue.

The antimicrobial metal can be applied as a layer by any of a number of methods, such as sputter coating, spray coating, or dip coating. However, in one specific example, the antimicrobial metal layer can be applied by electroless deposition. With this process, continuous layers of atomic metal, such as copper or copper alloy, can be electrically applied on an implantable medical device at or along a transcutaneous portion, at or along another portion susceptible to infection within the body, or over the entire implantable medical device. The thin layer can be applied so that the implantable medical device, where desired, can retain some of its flexibility. In other examples, the thickness can be such that the medical device becomes more rigid. There may be occasions to select thicker, more rigid coatings, and other occasions to select thinner coatings, depending on the application.

Electroless deposition works well on metal or conductive substrates. However, it can be useful to electroplate plastics, rubbers, ceramics, and/or other non-conductive, semi-conductive, or insulating materials. For example, electroless deposition chemistry allows electrically inert substrates such as plastics, rubbers, thermoplastics, and/or polymers to be plated with conductive metals facilitated by in-solution electrochemistry. Using copper electroplating as an example, Formula I shows an example chemical equation for electroplating a non-conductive material, as follows:

CuY²⁻+2CH₂O+4OH⁻→Cu⁰+H₂+2HCOO⁻+Y⁴⁻+2H₂O Formula 1

where Y represents a copper anion, such as sulfate, chloride, or phosphate; and Cu⁰ represents elemental copper electroplated on the surface of the substrate being plated, e.g., the implantable medical device.

In further detail, polymeric substrates can be employed in electroless deposition applications by treating the surface to make it adherent, e.g., cleaning, etching, and/or roughing, etc., and then the polymeric substrate can be dipped in a bath of a divalent metal, such as copper or nickel, for example, to give it a very thin coating of electrically conducting metal (typically less than 1 μm). There are other dipping, sputtering, and/or spraying methodologies that can be used to establish thicker layers, such as by multiple dip coatings or multi-coat spraying layers so that the antimicrobial metal “layer” is actually multiple layers of metal. Thus, the term “layer” includes any arrangement of metals as a single layer or as multiple adjacent layers that function as a “layer” of metal.

If electroplating the thin metal coating applied previously, the thin layer of metal can be electroplated similar to more conventional methods where a metal base is electroplated, e.g., with a metal ion source and a charge carrier while applying electrical potential to the charge carrier bath. Depending on the physical properties of the antimicrobial metal layer desired, the electroplating can occur to leave a layer that is from wear and tear that the plated part has to withstand, the coating can be anything from 0.0001 μm to 50 μm, though thinner layers may provide more implantable medical device flexibility when that is a consideration.

By using electroplating/electroless deposition to apply an antimicrobial metal to an implantable medical device, there can be several advantages achieved. For example, the electroplating can be carried out so that most or essentially all of the elemental metal from the metal source can be made available to the surface of the substrate to impart a higher degree of killing power, and in the case of copper, a higher degree of contact killing potential. This can also help bind the metal tightly to the substrate, even to a polymeric substrate or other non-conductive substrate with negligible leaching, e.g., “effectively non-leaching” levels as defined previously. Electroplating/electroless deposition can also provide for the application of a more precisely controlled application of the thin metal layer, which enables surface modification without compromising device features, e.g. flexibility, comfort, etc. The nature of electroless chemistry incorporates layers of the metal on an atom-by-atom level with areas exposed getting coated, including deformities, cracks, crevices, etc. This can prevent bacterial colonization at the surface, rather than permit colonization followed by killing through ion diffusion. These types of metal layers can also provide a consistent cover of substrate surfaces that may be inherently non-uniform, e.g., silicone.

The antimicrobial metal can be applied to the exterior surface of the medical device using methodologies other than forming a metal layer, as described above. For example, the antimicrobial metal can be provided in the form of an antimicrobial metal fiber or filament positioned at least partially at the exterior surface. The fiber or filament can be a fiber or filament coated with the antimicrobial metal, or the fiber or filament can be formed from, e.g., core to surface, of the antimicrobial metal. In one example, the antimicrobial metal fiber or filament includes copper. In another example, the antimicrobial metal fiber or filament is woven with a structural fiber or filament of the antimicrobial implantable medical device, e.g., polyester (or other polymer) fibers found on some drivelines used for various medical devices. The antimicrobial implantable medical device can thus be, or include, a driveline for an internal device that is powered externally.

As the surface of the implantable medical device is coated to include an antimicrobial metal, the metal can be activated by passing a small electrical potential across the surface to enhance, in some instances, the efficacy and strength of the antimicrobial performance.

In another example, a protective coating may be applied on top of the antimicrobial metal layer, such as a wax, a hydrophilic polymer, a hydrophobic polymer, etc., to prevent corrosion, for storage purposes, or even for therapeutic purposes. Likewise, the metal can be alloyed, as described previously, for any of a number of purposes, including preventing metal corrosion.

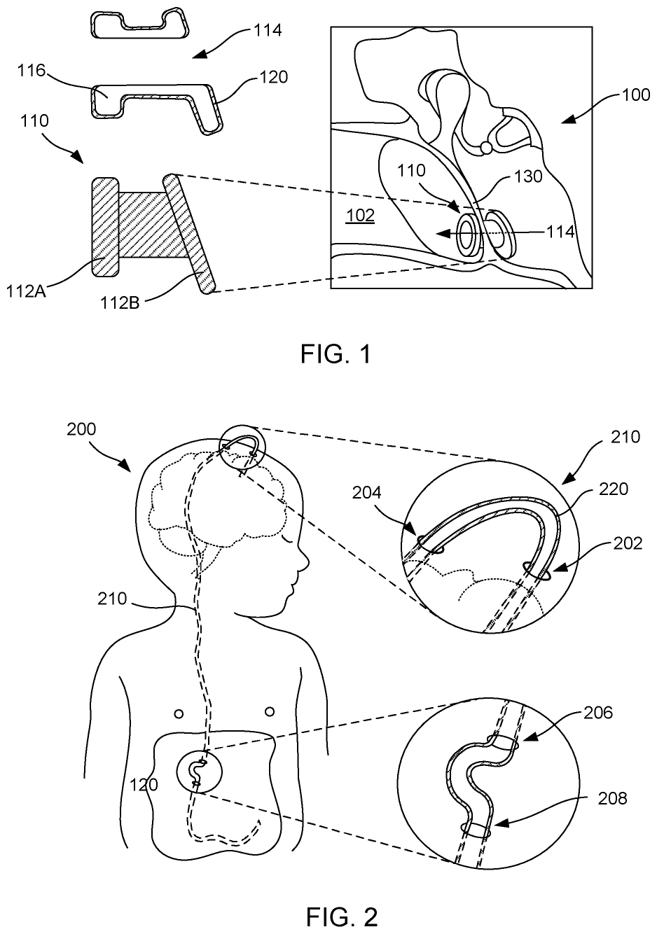

Referring now to FIG. 1, an example medical device coated with an antimicrobial metal layer 110 is shown that is surgically implantable within an ear 100 of a subject. More specifically, the medical device, a tympanostomy tube (ear tube) substrate material 116 to which the thin antimicrobial metal layer 120 is applied, as shown in this example, is a grommet style, double-flanged ear tube. The ear tube, prior to applying the thin metal layer can be, for example, metal, polymer, or any other material used for ear tubes. More specific examples include silicone, fluoropolymer or polyurethane, for example. The antimicrobial metal layer can be, for example, copper, silver, zinc, gold, or an alloy thereof. In one example, the thin metal layer is copper or a copper alloy. The thin metal layer can be as described previously with respect to material, layer thickness, application process, etc. Ear tubes are surgically implanted in the ear anatomy by creating an incision across the tympanic membrane (or ear drum) 130, and placing the flange 112A and 112B on either side of the ear tube (in the case of double-flanged ear tubes). Some ear tubes do not include multiple flanges or are not grommet style ear tubes, and thus they can be surgically implanted as advised.

Ear tubes 110 are inserted surgically to alleviate or prevent ear infections. However, ear tubes are also sometimes the cause of secondary infection, in the form of postoperative otorrhea where pus formation or fluid accumulation occurs at the implantation site. This can lead to ear tube malfunction and recurrent infection symptoms. By coating the ear tube with an antimicrobial metal layer 120, these infections can be reduced.

Though the ear tube 110 shown in FIG. 1 is an asymmetrical beveled grommet type ear tube having a configuration similar to the Armstrong beveled grommet, any other type of ear tube can be used as well, such as Shepart Grommets, Soileau Tytan Titanium Ventilation Tubes, Spoon Bobbins, Goode T-Tubes, Paparella-type vent tubes, triune tubes, or the like. Grommets or other shaped-ear tubes can be beveled or non-beveled with a flange or flange-like structure 112A on one side, and more typically a flange or flange-like structure 112B on the other side as well of the same or different shape or configuration. The ear tubes also include a drainage tube 114 therethrough to allow the antimicrobial implantable medical device to facilitate air flow and fluid drainage from the middle ear 104 into the external auditory canal 102. Most grommets or other types of ear tubes are for short term use, e.g., 6 to 12 months, but may be implanted for 36 months or longer. Even short duration ear tube implants are relatively long duration implants with respect to the potential for colonization of bacteria thereof. Thus, the antimicrobial metal layers described herein can ameliorate possible secondary infections.

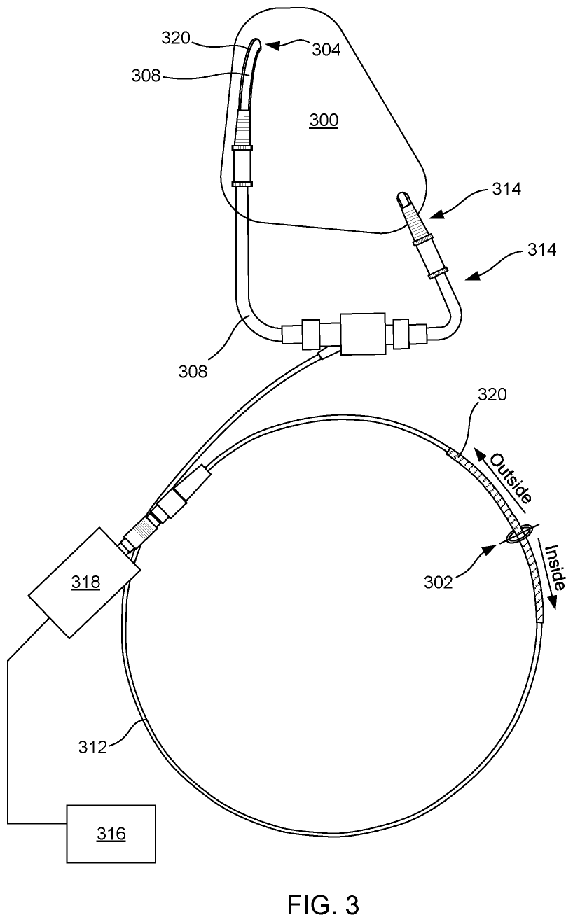

In another example, as shown in FIG. 2 by example, the antimicrobial implantable medical device that can be implanted in a subject 200 can be a shunt 210, such as a cerebral shunt, which is a device that is used to divert bodily fluids from one place in the body to another. The shunt can be any medically implanted shunt to transport fluids from one location in the body to another. In this example, a cerebral shunt is shown that can be coated with a thin antimicrobial metal layer 220, such as copper, silver, zinc, gold, or an alloy thereof. In one example, the thin metal layer is copper or a copper alloy. The thin metal layer can be as described previously with respect to material, layer thickness, application process, etc. Alternatively, the medical device can be a medical drainage tube, such as an EVS, that diverts fluids from the body to an external site for collection.

In one example, the shunt 210 can be a cerebral shunt, which is a transcutaneous device that can be subject to infection where the shunt exits the brain and head 202 and enters 204 again to be ported beneath a body surface to a drainage location where the cerebral shunt may exit 206 and enter 208 the body at or near a drainage location. In other examples not shown, cerebral shunts can exit the brain one time and re-enter the drainage location one time, or can exit and re-enter at or near the head, and then be passed beneath the body surface to enter the drainage location. Example cerebral shunts that can be used to drain cerebral spinal fluid (CSF) from any of the ventricles to elsewhere in the body include ventriculoperitoneal (VP) shunts (draining to the peritoneal cavity), ventriculoatrial (VA) shunts (draining to the right atrium of the heart), ventriculopleural (VPL) shunts (draining to the pleural cavity), ventriculocisternal (VC) shunts (draining to the cisternal magna), ventriculosubgaleal (SG) shunts (draining to the subgaleal space), lumboperitoneal (LP) shunts (draining to the peritoneal cavity), etc.

Similar to cerebral shunts, external ventricular drains (EVDs) can also be used to drain cerebral spinal fluid (CSF) from any ventricle of the brain, but the fluid is drained outside of the body rather than within the body. Thus, EVDs are also considered to be implantable medical devices in accordance with the present disclosure, as they are implanted surgically and are not considered to be luminal medical devices as described herein.

Cerebral shunts of various types, as well as EVDs, can be used to manage conditions such as hydrocephalus. These types of devices fail in the first year about 40% of the time, with an overall failure rate of about 50%. Infection is one of the major reasons for failure. For example, the region of the device the shunt exits and enters the head (at the brain) or other body location is susceptible to microbial ingress. Commercially available antimicrobial systems include the Medtronic ARES, which is an antibiotic coated EVD. Other antimicrobial protection systems include the J&J Codman system and the Cook Spectrum system, both of which are not very effective for longer term use. Furthermore, many antibiotics used in these types of cerebral shunts and/or EVDs use antibiotics such as Minocycline/Clindamycin and Rifampin that are known to be ineffective against some bacterial strains (gram negative and yeast). Even with these antibiotic coatings, mortality and morbidity rates still remain high, e.g., Pediatric Cerebral Shunts have a 6-25% morbidity and about a 2.7% mortality rate with over a $35,000 treatment cost per instance. The antimicrobial metal layers applied to shunts, surgically implanted drainage tubes such as EVDs, or the like can provide an alternative to antimicrobial colonization intervention, and may work well against antibiotic resistance bacterial strains.

In another example, and as shown in FIG. 3 by example, the implantable medical device can be a ventricular assist device (VAD) 310 connected to the heart 300 of a subject, or a component thereof, such as the driveline 312, or a component of the pump 314 positioned within the peritoneal cavity. A power supply 316 and a controller 318 are positioned outside of the body and can be used to power and control the pump. Thus, the driveline in particular is placed transcutaneously and can be susceptible to bacterial migration and colonization. Local and driveline infections are high in this long term implant, with infection rates reported to be as high as 60%, which is treated primarily with antibiotic therapies when infections occur, and local site disinfection for maintenance. Sometimes a second surgical intervention is needed to combat the infection. These types of implantable medical devices can also lead to pump pocket infection (in the body space where the pump is located) which is hard to discover without surgical intervention, and/or even bloodstream infections that can be associated with stroke and mortality,

Thus, in accordance with the present disclosure, the ventricular assist device (VAD) 310, or component thereof (driveline 312, pump tubing 308, fluidic fittings 314, or the like), can be coated with a thin antimicrobial metal layer 320, such as copper, silver, zinc, gold, or an alloy thereof to reduce the impact or chances that a subject will get an infection at the transcutaneous site, or at other sites where the device crosses or intimately contacts body tissues or organs. In one example, the thin metal layer can be copper or a copper alloy. The thin metal layer can be as described previously with respect to material, layer thickness, application process, etc. The thin metal layer, as shown likewise in previous examples, can be present at least at one location, and sometimes multiple locations, where the medical device is inserted within or traverses a body tissue, such as the skin 302 or heart muscle 304, as shown by example and not limitation.

There are also other implantable medical devices that can be coated with the antimicrobial metal layers of the present disclosure, but which are not specifically shown in the FIGS. However, these implantable medical devices, or components thereof (which are also defined to be “implantable medical devices”) can likewise be coated with a thin antimicrobial metal layer, such as copper, silver, zinc, gold, or an alloy thereof to reduce the impact or chances that a subject will get an infection at the transcutaneous site, or at other sites where the device crosses or intimately contacts body tissues or organs. As mentioned, the thin metal layer can be copper or a copper alloy, for example. The thin metal layer can be as described previously with respect to material, layer thickness, application process, etc.

Examples of such devices include orthopedic implants, which can be used to treat bone fractures, osteoarthritis, scoliosis, spinal stenosis, chronic pain, joint replacement, etc. As mentioned, simple surgical fasteners, such as pins, wires, staples, screws, nails, etc. are not considered to be implantable medical devices in accordance with the present disclosure. The orthopedic implants that can be coated with an antimicrobial metal layer in accordance with the present disclosure may otherwise be susceptible to bacterial colonization as a result of the surgery, or even bacterial colonization introduced after surgery. Staphylococci, particularly Staphylococcus aureus or Staphylococcus epidermidis, are particularly prevalent with orthopedic implants. Bacterial infection can lead to, for example, bone tissue infections such as osteomyelitis, septic arthritis, or prosthetic joint infections (PJI). Pathways for infection can be septic arthritis, hematogenous (bacteria introduced in blood then introduced to local tissue at the site of the implant), and bone infiltration (from surgery, injury, etc.). As orthopedic implants are of many different types of materials, bacterial adhesion can be problematic with some materials for certain types of bacteria. With adhesion at the implant site, bacterial colonization can occur at the surface, and some bacteria can secrete a layer of slime or form a biofilm that is self-protecting. By applying the antimicrobial thin metal layers to implantable devices as described herein, the surfaces that may be susceptible to adhesion can be reduced due to particularly contact killing that can occur with copper layers.

Example implantable medical devices (along with where they are implanted) that can be coated with an antimicrobial thin metal layer include, but are not limited to: Austim-Moore prostheses (neck of femur), Baksi's prostheses (elbow), Charnley prostheses (hip), Harrington rods (spine), Hartshill rectangles (spine), Insall Burstein prostheses (knee), Richard N. W. Wohns interspinous implant and implantation instrument (between adjacent dorsal vertebrae), Luque rod (spine), Neer's prostheses (shoulder), McLaughlin's plate (inter-trochanteric fracture), Souter's prosthesis (elbo), Steffee plate (spine), Swanson prostheses (finger joint), Thompson prosthesis (neck of femur), etc. There are many other prosthetic devices that can be coated with copper or another antimicrobial metal in accordance with the present disclosure to prevent bacterial adhesion and colonization.

Other implantable medical devices that can be coated with an antimicrobial metal layer include sensory or neurological implants, such as those for treating disorders of the brain or other neurological systems. Surgically implantable medical devices for the treatment of hearing loss, such as otosclerosis, can be coated with an antimicrobial metal layer, including devices at common infection locations of hearing restoration implant devices, e.g., cochlear implants. Otitis media can be treated with ear tubes, as previously described. Electrical devices, such as neurostimulators, can likewise be implanted to stimulate the brain or other neurological systems to treat any of a number of neurological diseases, such as epilepsy, Parkinson's disease, treatment-resistant depression, etc. In these instances, for example, the lead wires and/or other transcutaneous or trans-tissue structures can likewise be coated with copper or other antimicrobial metal as described herein at locations susceptible to infection.

Cardiovascular implantable medical devices (excluding vascular catheters as defined herein, for example) are another category of devices that can benefit from the antimicrobial metal layers described herein. These types of devices can be used to treat heart failure, cardiac arrhythmia, ventricular tachycardia, valvular heart disease, angina pectoris, atherosclerosis, etc. Example implantable medical devices considered to be cardiovascular include the artificial heart, the artificial heart valve, (implantable) cardioverter-defibrillator, cardiac pacemaker, sensors such as glucose sensors, etc.

It is to be noted that, as used in this specification and the appended claims, the singular forms “a,” “an,” and “the” include plural referents unless the context clearly dictates otherwise.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the description herein.

Sizes, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1.0 to 2.0 percent” should be interpreted to include not only the explicitly recited values of about 1.0 percent to about 2.0 percent, but also include individual values and sub-ranges within the indicated range. Thus, included in this numerical range are individual values such as 1.1, 1.3, and 1.5, and sub-ranges such as from 1.3 to 1.7, 1.0 to 1.5, and from 1.4 to 1.9, etc. This same principle applies to ranges reciting only one numerical value. Furthermore, such an interpretation should apply regardless of the breadth of the range or the characteristics being described.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

It is also noted that any of the device features described herein and/or shown in the FIGS. can be combined together in any manner that is not specifically shown or described. For example, it is not the purpose of the present disclosure to put together every possible combination of features in the drawings or description, but rather to describe fully the combination of concepts to be combined with various types.

### EXAMPLES

The following illustrates several examples of the present disclosure. However, it is to be understood that the following examples are only illustrative of the application of the principles of the present disclosure. Numerous modifications and alternative structures, compositions, methods, and systems may be devised without departing from the present disclosure. The appended claims are intended to cover such modifications and arrangements.

### Example 1—Tympanostomy Tube (Ear Tube) Bacterial Colonization Study 1

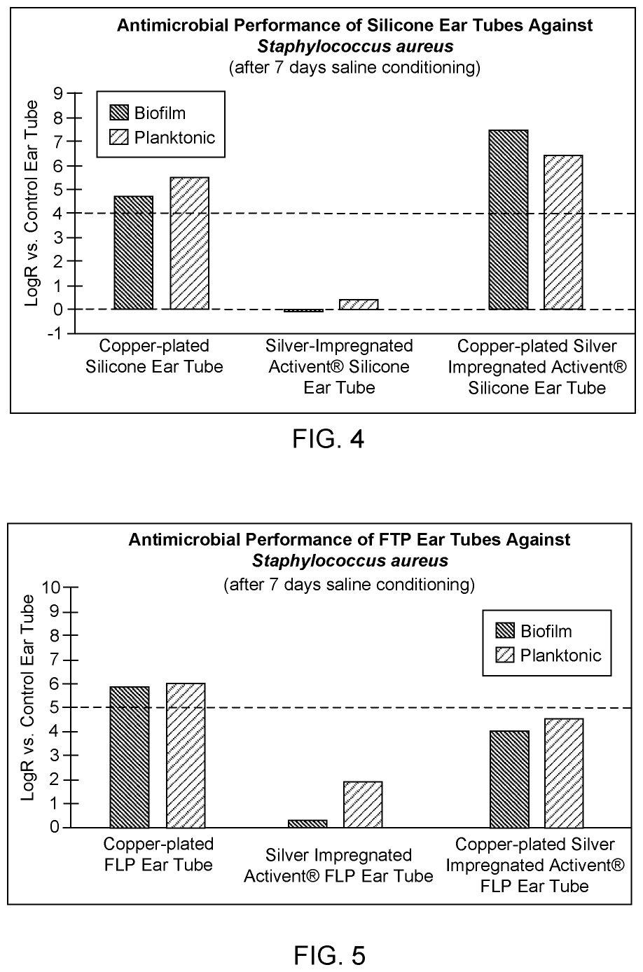

In vitro antimicrobial performance of copper-plated silicone ear tubes was compared to uncoated untreated silicone ear tubes, where the copper-plated silicone antimicrobial ear tubes were characterized for performance based on a log-scale reduction scale (Log R). Thus, the higher the Log R value, the lower level of bacterial colonization because the high value indicates a higher level of improvement against the untreated silicone ear tubes. In this study, both types of ear tubes were exposed to Staphylococcus aureus. At the same time for comparison, the performance of commercially available antimicrobial ear tubes, namely silver oxide impregnated Activent® ear tubes was also similarly studied and characterized in terms of Log R. Furthermore, a second set of Activent® ear tubes was also obtained and coated with the copper-plating antimicrobial layer similar to that described above. All three ear tube groups in the study were exposed to daily saline change outs for 7 days prior to microbial challenge. The data collected was based on a bacterial challenge where a concentration ˜1×10⁴ CFU/mL of bacteria was exposed to the ear tubes, and the colonies were allowed to incubate at 37° C. in in the presence of saline-based nutrient broth for 24 hours. Both biofilm (adherent bacteria) and planktonic (free floating bacteria) recoveries from the respective devices and surrounding media is shown in FIG. 4.

As can be seen in FIG. 4, the copper coated ear tubes (both with and without the Activent® technology) outperformed the Activent® ear tubes without a copper coating applied thereto with respect to biofilm colonization.

### Example 2—Tympanostomy Tube (Ear Tube) Bacterial Colonization Study 2

The same study of Example 1 was conducted, except that instead of silicone ear tubes, fluoropolymer (FLP) based ear tubes were used instead. Both biofilm (adherent bacteria) and planktonic (free floating bacteria) recoveries from the respective devices and surrounding media is shown in FIG. 5.

As can be seen in FIG. 5, the copper coated ear tubes (both with and without the Activent® technology) significantly outperformed against the Activent® ear tubes both with respect to planktonic and biofilm colonization.

### Example 3—External Ventricular Drain (EVD) Bacterial Colonization Study 1

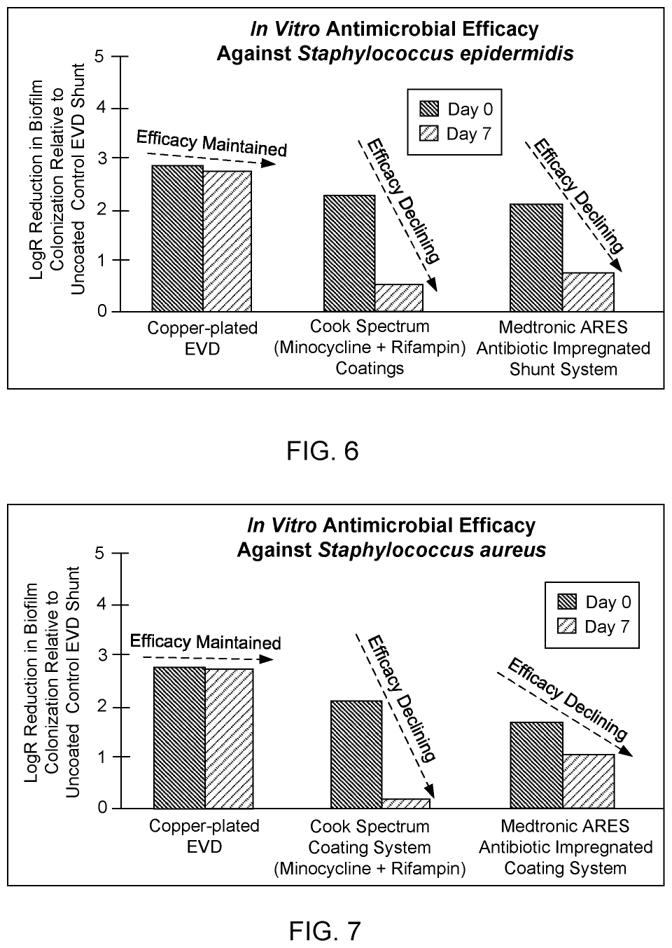

In vitro antimicrobial performance of a copper-plated silicone EVD was compared to an uncoated untreated silicone EVD, where the copper-plated EVD was characterized for performance based on a log-scale reduction (Log R). Thus, the higher the Log R value, the lower the level of bacterial colonization because the high value indicates a higher level of improvement against the untreated silicone EVD. In this study, both types of EVD tubing were exposed to Staphylococcus epidermidis. At the same time for comparison, the performance of two commercially available antimicrobial coating systems were also evaluated, namely the Cook Spectrum coating on EVD available from Cook Medical (USA) and the ARES® antibiotic-impregnated coating system from Medtronic (USA). The data collected was based on a bacterial challenge where a concentration ˜1×10⁶ CFU/mL of bacteria was exposed to the devices, and the colonies were allowed to incubate at 37° C. in in the presence of saline-based nutrient broth. Only biofilm (bacterial adherent) recoveries were collected, but were collected after Day 0 (initial) and Day 7 (1 week later) exposure to daily saline changeouts. Both time points were exposed to 24 h of microbial challenge described above. The data collected is shown by way of example in FIG. 6.

As can be seen, the copper coating technology of the present disclosure showed at least equivalent antimicrobial efficacy to both of the antibiotic catheters at Day 0, but the copper coating also effectively retained antimicrobial efficacy after 7 days of simulated conditioning, while both antibiotic catheters had a significantly reduced efficacy.

### Example 4—External Ventricular Drain (EVD) Bacterial Colonization Study 2

The same study of Example 3 was conducted, except that instead of challenging the medical devices with Staphylococcus epidermidis, the devices were challenged with Staphylococcus aureus. The data collected is shown by way of example in FIG. 7. As can be seen, the copper coating technology of the present disclosure showed at least equivalent antimicrobial efficacy to both of the antibiotic catheters at Day 0, but the copper coating also effectively retained antimicrobial efficacy after 7 days of simulated conditioning, while both antibiotic catheters had a significantly reduced efficacy.

### Example 5—External Ventricular Drain (EVD) Bacterial Colonization Study 3

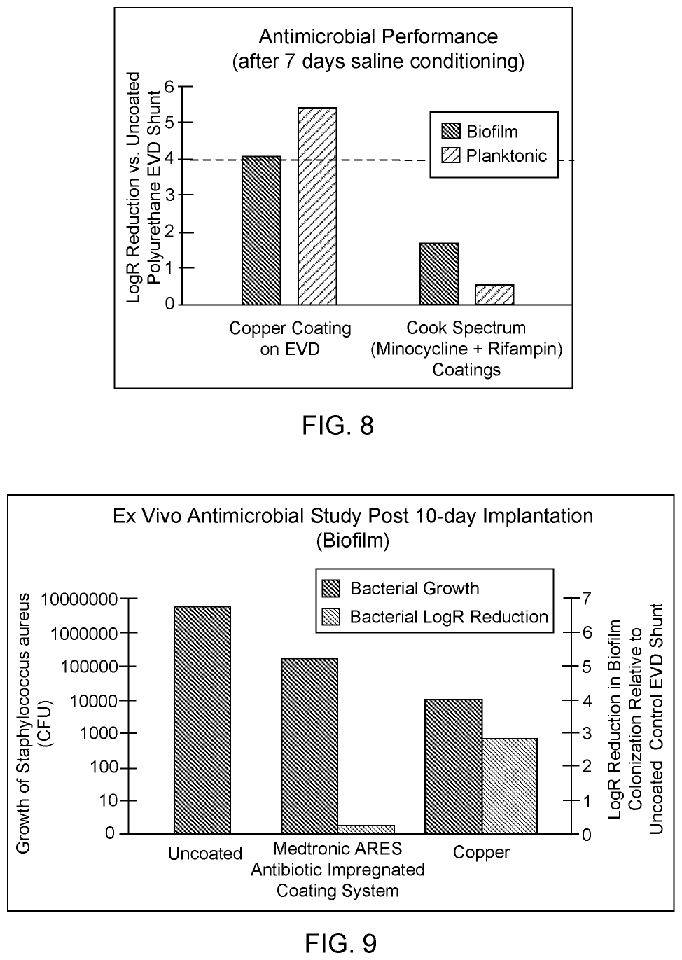

In vitro antimicrobial performance of a copper-plated polyurethane EVD was compared to an uncoated and untreated polyurethane EVD, where the copper-plated silicone antimicrobial EVD was characterized for performance based on a log-scale reduction (Log R). Thus, the higher the Log R value, the lower the level of bacterial colonization because the high value indicates a higher level of improvement against the untreated silicone EVD. In this study, both types of EVDs were exposed to Staphylococcus aureus. At the same time for comparison, the performance of a commercially available antimicrobial coating system was evaluated, namely the Cook Spectrum coating from Cook Medical (USA). All medical devices in the study were exposed to daily saline change outs for 7 days prior to microbial challenge. The data collected was based on a bacterial challenge where a concentration ˜1×10⁴ CFU/mL of bacteria was exposed to the ear tubes, and the colonies were allowed to incubate at 37° C. in the presence of saline-based nutrient broth fluid for 24 hours. Both biofilm (adherent bacteria) and planktonic (free floating bacteria) recoveries from the respective devices and surrounding media is shown in FIG. 8. As can be seen in FIG. 8, the copper coated EVD outperformed the medical device with the Cook Spectrum system coated thereon.

### Example 6—In Vivo Biocompatibility of External Copper Plating and Ex Vivo Antimicrobial Efficacy Study

A pilot rabbit study was conducted to determine the tolerance of the animal to exposure to the copper plating, e.g., to determine if there would be a biological reaction to the copper to identify if there would be tolerance at a site of a surgically generated incision site or as an implanted device. For this study, copper-plated shunt devices as well as devices coated with the ARES® antibiotic-impregnated coating system, from Medtronic (USA), were implanted subcutaneously into pockets of the skin of a rabbit. The shunt material that was evaluated for this study was silicone. For comparison, uncoated shunts were also implanted in the same rabbit. The shunts were left in the rabbit for 10 days to understand the biological reaction to the new material, and then extracted from the animals to evaluate the antimicrobial effect of the copper plating vs. the ARES® antibiotic-impregnated coating system vs. uncoated devices under in vitro conditions. To evaluate solely the outside of the medical devices for the antimicrobial study, the inside of the shunts were sealed off with epoxy so that the evaluation was solely related to the exterior of the implanted device.

The shunt devices extracted from the animals were inoculated with bacterial cultures and incubated under in vitro conditions, e.g., on a rotary shaker at 37° C. The copper-plated shunts exhibited almost a Log 3 reduction in growth of Staphylococcus aureus compared to the uncoated device, whereas the ARES® antibiotic-impregnated coating system was only slightly better, e.g., less than a Log 1 reduction, compared to the uncoated device. The raw data for the study is shown in FIG. 9, which shows there were close to 10,000,000 microbes on the uncoated shunts, whereas the copper-plated shunt device retained only about 10,000 microbes. This study also indicated that there were no signs of toxicity to the animal, as verified by histopathology work done on the rabbit tissue. In addition monitoring for signs of toxicity such as local inflammation, redness of skin, pus formation, fever, loss of weight, etc. also indicated no gross signs of toxicity.

While the forgoing examples and descriptions are illustrative of the principles of the present technology in one or more particular applications, it will be apparent to those of ordinary skill in the art that numerous modifications in form, usage and details of implementation can be made without the exercise of inventive faculty, and without departing from the principles and concepts of this technology. Accordingly, it is not intended that the technology be unduly limited.

What is claimed is:

1. An antimicrobial implantable medical device, comprising:
an implantable medical device; and
an antimicrobial metal applied to an exterior surface of least a portion of the implantable medical device that is positionable i) within a body tissue or ii) traversing the body tissue, when surgically placed for implantation.

2. The antimicrobial implantable medical device of claim 1, wherein the antimicrobial metal is applied to the exterior surface by electroless deposition.

3. The antimicrobial implantable medical device of claim 1, wherein the antimicrobial metal is an antimicrobial metal fiber or filament positioned at least partially at the exterior surface.

4. The antimicrobial implantable medical device of claim 3, wherein the antimicrobial metal fiber or filament includes copper.

5. The antimicrobial implantable medical device of claim 3, wherein the antimicrobial metal fiber or filament is woven with a structural fiber or filament of the antimicrobial implantable medical device.

6. The antimicrobial implantable medical device of claim 5, wherein the antimicrobial implantable medical device is a driveline for an internal device that is powered externally.

7. The antimicrobial implantable medical device of claim 1, wherein the antimicrobial metal layer comprises copper, silver, zinc, gold, tin, or an alloy thereof.

8. The antimicrobial implantable medical device of claim 1, wherein the antimicrobial metal is elemental copper.

9. The antimicrobial implantable medical device of claim 1, wherein the antimicrobial metal is an alloy of elemental copper and one or more additional metals.

10. The antimicrobial implantable medical device of claim 1, wherein the implantable medical device is a surgically-implantable transcutaneous device.

11. The antimicrobial implantable medical device of claim 1, wherein the implantable medical device is a surgically-implantable device that is not transcutaneous, residing when implanted completely beneath a skin surface of the body of the subject.

12. The antimicrobial implantable medical device of claim 1, wherein the implantable medical device is a tympanostomy tube.

13. The antimicrobial implantable medical device of claim 1, wherein the implantable medical device is a shunt.

14. The antimicrobial implantable medical device of claim 13, wherein the shunt is a cerebral shunt.

15. The antimicrobial implantable medical device of claim 1, wherein the implantable medical device is an external ventricular drain.

16. The antimicrobial implantable medical device of claim 1, wherein the implantable medical device is a ventricular assist device.

17. The antimicrobial implantable medical device of claim 1, wherein the implantable medical device is an orthopedic implant.

18. The antimicrobial implantable medical device of claim 1, wherein the implantable medical device is a sensory or neurological implant.

19. The antimicrobial implantable medical device of claim 1, wherein the implantable medical device is a sinus drainage device.

20. The antimicrobial implantable medical device of claim 1, wherein the implantable medical device is a cochlear implant.

21. The antimicrobial implantable medical device of claim 1, wherein the implantable medical device is a hernia mesh.

22. The antimicrobial implantable medical device of claim 1, wherein the implantable medical device includes an electrical power or control line positionable within the body tissue or traversing the body tissue.

23. The antimicrobial implantable medical device of claim 1, wherein the implantable medical device is a cardiovascular implant.

24. The antimicrobial implantable medical device of claim 1, wherein the antimicrobial metal layer is an electroplated metal having a thickness from 0.0001 μm to 50 μm.

25. The antimicrobial implantable medical device of claim 1, wherein the antimicrobial metal layer is an electroplated metal having a thickness from 0.001 μm to 0.1 μm.

26. The antimicrobial implantable medical device of claim 1, wherein the implantable medical device is at a location where it is within the body tissue or traverses the body tissue is of a polymeric material, and wherein the antimicrobial metal layer at the location is applied via electroless deposition.

27. The antimicrobial implantable medical device of claim 1, wherein the implantable medical device is at a location where it is within the body tissue or traverses the body and tissue includes the antimicrobial metal layer thereon at least 0.5 cm on one side of the body tissue.

28. The antimicrobial implantable medical device of claim 1, wherein the implantable medical device is at a location where it traverses the body tissue and includes the antimicrobial metal layer thereon at least 0.5 cm on both sides of the body tissue traversed.

29. The antimicrobial implantable medical device of claim 1, wherein the implantable medical device in operation includes a lumen that not subjected to externally applied positive or negative pressure while in use after implantation.

30. A method of ameliorating implantable medical device-related infection, comprising surgically placing the implantable medical device of any one of claims 1 to 28 at least partially within the body of a subject, wherein the antimicrobial metal layer of the medical device is positioned for implantation at a location of within the body tissue or traversing a body tissue.

31. The method of ameliorating implantable medical device-related infection of claim 30, further comprising applying an electrical potential to the antimicrobial metal layer while the microbial metal layer is positioned at the location.

32. The method of ameliorating implantable medical device-related infection of claim 30, wherein the implantable medical device is a transcutaneous device, and the method further comprises leaving a margin of at least 0.5 cm outside of the body beyond the location of insertion through the cutaneous tissue.

33. A method of manufacturing the antimicrobial implantable medical device of any one of claims 1 to 29, comprising applying a metal layer on the implantable medical device by electroless deposition at least at a location where the implantable medical device is to be positioned within the body tissue or traversing a body tissue for implantation.

34. A method of manufacturing the antimicrobial implantable medical device of claim 33, wherein the electroless deposition is carried out using a copper salt source material which in solution is reduced to metallic copper in the presence of a reducing agent which in turn gets oxidized and the metallic copper atoms are deposited on any surface in the bath including the medical device surfaces to generate a copper or copper alloy antimicrobial metal layer.

35. The method of claim 34, wherein the medical device has a polymer surface and wherein the method further includes a preliminary step of introducing surface roughness to the polymer surface.

36. The method of claim 34, wherein introducing surface roughness includes chemical etching, mechanical abrasion, physical etching, or plasma treatment.
